(19) United States
(12) Patent Application Publication
(10) Pub. No.: US 2023/0121926 A1
(43) Pub. Date: Apr. 20, 2023

(54) METHODS FOR TISSUE REGENERATION AND KITS THEREFOR

(71) Applicant: THE BOARD OF TRUSTEES OF THE LELAND STANFORD JUNIOR UNIVERSITY, Stanford, CA (US)

(72) Inventors: Geoffrey C. Gurtner, Stanford, CA (US); Kellen Chen, Stanford, CA (US); Sun Hyung Kwon, Stanford, CA (US); Dominic Henn, Stanford, CA (US)

(73) Assignee: THE BOARD OF TRUSTEES OF THE LELAND STANFORD JUNIOR UNIVERSITY, Stanford, CA (US)

(21) Appl. No.: 17/914,134

(22) PCT Filed: Jan. 25, 2021
(86) PCT No.: PCT/US2021/014847

§ 371 Date: Sep. 23, 2022

Related U.S. Application Data
(60) Provisional application No. 63/000,309, filed on Mar. 26, 2020.

(51) Int. Cl. A61L 27/54; A61L 27/24; A61L 27/52; A61L 27/56

(52) U.S. Cl. CPC A61L 27/54; A61L 27/24; A61L 27/52; A61L 27/56; A61L 2430/18; A61L 2430/34; A61L 2300/412; A61L 2300/602

(57) ABSTRACT

Methods are described herein for facilitating tissue regeneration in humans and other large organisms, and kits therefor. Application of an inhibitor of focal adhesion kinase (FAK) to injured tissue may reduce fibrosis and/or scarring during the wound healing process. Patient care for a large number of fibrotic diseases which affect organ function may be ameliorated by such treatment. Kits for application of the FAK inhibitor may include a hydrogel formulation encapsulating the FAK inhibitor.

### CROSS-REFERENCE TO RELATED APPLICATIONS

This application is the national phase entry of PCT application PCT/US2021/014847, filed Jan. 25, 2021, which claims the benefit of priority of U.S. Provisional Application No. 63/000,309, filed Mar. 26, 2020, entitled “METHODS FOR TISSUE REGENERATION AND KITS THEREFOR”. Each of the above applications is incorporated by reference as if fully set forth herein.

### STATEMENT AS TO FEDERALLY SPONSORED RESEARCH

This invention was made with Government support under contract DE026914 from awarded by the National Institutes of Health. The Government has certain rights in the invention.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

In humans and other large organisms, tissue injury results in scar formation and fibrosis. This is in contrast to tissue repair that occurs in planaria, certain mice, and other “model organisms” in which injury leads to regeneration of normal tissue architecture with no fibrosis. Facilitating tissue regeneration in humans and other large organisms is one of the “holy grails” of biomedical research and could revolutionize patient care for a large number of fibrotic diseases which affect organ function. Such diseases may include but are not limited to myocardial infarction and ischemic stroke, each of which have significant economic and quality of life impact for individuals and for society at large. Additionally, reduction of fibrotic scarring from traumatic injuries, including burns, blunt and penetrating wounds to skin and underlying tissue would be a significant improvement to outcome in such instances. Novel approaches to ameliorate fibrotic/scar formation are needed.

### SUMMARY OF THE DISCLOSURE

The present invention relates to methods and kits for promoting wound healing while reducing fibrosis and/or scarring in a large mammal, such as a human, which includes administering a composition including a focal adhesion kinase (FAK) inhibitor proximally to a wounded tissue of the large mammal. The FAK inhibitor may be locally administered. The composition may include a porous scaffold, where the FAK inhibitor is disposed within the pores of the porous scaffold.

In a first aspect, a method of promoting tissue healing while reducing fibrosis in a large mammal is provided including: disposing a composition containing an effective amount of a focal adhesion kinase (FAK) inhibitor in proximity to a tissue of the large mammal, where the tissue includes a wound; dispensing the FAK inhibitor from the composition into the proximity of the wounded tissue; and reducing a level of focal adhesion kinase for a selected period of time, thereby reducing fibrosis while healing the wounded tissue.

In some variations, the composition containing the FAK inhibitor may be administered locally.

In some variations, the composition may include a porous scaffold and the FAK inhibitor is disposed in pores of the porous scaffold. In some variations, the porous scaffold may include a hydrogel. In some variations, the porous scaffold hydrogel may be a thin film. In some variations, the hydrogel may be a pullulan-collagen hydrogel.

In some variations, the large mammal may be a human.

In some variations, the FAK inhibitor may be VS-6062.

In some variations, the wound may be an incision, a penetrating wound, or a burn.

In some variations, the FAK inhibitor may be formulated for controlled release.

In some variations, the selected period of time for treatment with the composition containing the FAK inhibitor may be from about 7 days to about 100 days. In some variations, the composition containing the FAK inhibitor may be freshly applied to the proximity of the tissue every 36 to 48 hours.

In some variations, the effective amount of the focal adhesion kinase inhibitor may be from about 30 to about 100 micrograms/g tissue by weight.

In another aspect, a kit for promoting tissue healing while reducing fibrosis in a large mammal is provided, including: a composition containing a FAK inhibitor configured for local administration to a wounded tissue of the large mammal. In some variations, the composition containing the FAK inhibitor is configured to deliver about 30 to about 100 micrograms/g tissue by weight of the FAK inhibitor. In some variations, the composition containing the FAK inhibitor is configured to deliver the FAK inhibitor in a controlled release manner.

In some variations, the composition may include a porous scaffold and the FAK inhibitor is disposed in pores of the porous scaffold. In some variations, the porous scaffold may include a hydrogel. In some variations, the porous scaffold hydrogel may be a thin film. In some variations, the hydrogel may be a pullulan-collagen hydrogel.

In some variations, the kit may further include a wound dressing configured to protect the wounded tissue.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A. Disruption of mechanotransduction in large organisms accelerates deep partial-thickness wound healing, attenuates fibrotic scar formation, and promotes tissue regeneration. Large area (5×5 cm) deep partial-thickness excisional wounds (˜25 cm²) were created on the lateral dorsum (left and right) of red Duroc pigs (photograph of fresh wounds, bottom row). Wounds were either treated with: standard bandage dressings (Wounded: W, grey box, left); blank pullulan-collagen hydrogels (Wounded+hydrogel: W_H, blue box, center); or focal adhesion kinase inhibitor (FAKI)-releasing hydrogels (Wound+FAKI hydrogel: W_HF, red box, right) (total n=6-9 wounds per condition).

FIG. 1B. Disruption of mechanotransduction in large organisms accelerates deep partial-thickness wound healing, attenuates fibrotic scar formation, and promotes tissue regeneration. Wounds in all three groups were evaluated by gross photography at indicated time points until postoperative day (POD) 180. FAKI hydrogel treatment continued until POD 90. Dressing changes in all pigs continued until POD 90.

FIG. 1C. Disruption of mechanotransduction in large organisms accelerates deep partial-thickness wound healing, attenuates fibrotic scar formation, and promotes tissue regeneration. Wound closure rates and Visual Analog Scale (VAS) scoring was assessed by four blinded scar experts by quantification of digital photos of wounds throughout the healing process (wound closure assessed from POD 0 to POD 25; VAS assessed at POD 90). n=3 randomly selected image per wound.

FIG. 1D. Disruption of mechanotransduction in large organisms accelerates deep partial-thickness wound healing, attenuates fibrotic scar formation, and promotes tissue regeneration. Wound firmness (left) and elasticity (right) was compared between normal wounds (W) and FAKI-treated wounds (W_HF) by cutometer at POD 60 (n=8 wounds per condition). Statistical comparisons were made by using analysis of variance (ANOVA) using unpaired two-tailed t-tests (p<0.05).

FIG. 1E. Picrosirius Red staining of the three wound groups.

FIG. 1F. Three wound groups were quantified and compared to unwounded skin (UW) using strength of alignment (mean vector length; MVL, top graph) and fiber length metrics (pixels; px, bottom graph) (n=9 wounds/condition). Statistical comparisons were made by using analysis of variance (ANOVA) with Tukey's multiple comparisons tests (*p<0.05; **p<0.01;***p<0.001; ****p<0.0001).

FIG. 1G. Masson's Trichrome staining of healed scar (samples were collected from the center of the original excisional wounds) to assess the presence of hair follicles (yellow solid arrows), secondary cutaneous glands (black solid arrows), and intradermal adipocytes proximal to the appendage structures (yellow dashed arrows). Scale bar: 200 μm.

FIG. 1H. Blinded wound experts counted the hair follicles (top) and cutaneous glands (bottom) in healed wounds at POD 90 and POD 180. Statistical comparisons were made either by using analysis of variance (ANOVA) with Tukey's multiple comparisons tests (*p<0.05; **p<0.01;***p<0.001; ****p<0.0001).

FIG. 2A is a schematic representation of the process of isolating adult human dermal fibroblasts from tissue collected from three patients at different anatomical locations; the breast from a mastectomy sample, the abdomen from an abdominoplasty sample, and the thigh from a thighplasty sample. Freshly isolated fibroblasts were seeded into 3D collagen scaffolds and subjected to either no strain (NS, blue), strain (S, green), or strain and 10 μM FAKI (S+FAKI, orange) and then submitted for massively parallel sequencing (10× Genomics) and genomic analysis.

FIG. 2B. The collagen scaffolds were subjected to 10% strain and used titanium oxide dots (inner 9 circles) to track the exact imposed strains, shown by gross photography (top row) and a schematic (bottom row). Scaffolds were pinned at the arms to enforce the strain (outer circles; 2 circles per arm). Scale bar: 1 cm

FIG. 2C. UMAP density plots of cellular transcription profiles in no strain (NS, blue), strain (S, green), and strain+FAKI (S+FAKI, orange) groups.

FIG. 2D. Unsupervised clustering of fibroblast transcriptional signatures revealed a total of 8 distinct subpopulations of human dermal fibroblasts (numbered 0 to 7).

FIG. 2E. Heatmap of the top 5 differentially expressed genes in all clusters (left), and key pathways upregulated by the most differentially expressed genes in each cluster as revealed by gene ontology analysis (right).

FIG. 2F. Feature UMAP plots of cluster-defining differentially expressed genes shown with corresponding violin plots illustrating the expression levels per cluster; PTPN11—protein tyrosine phosphatase non-receptor type 11, MMP1—metalloproteinase 1, COL1A1—collagen type 1 alpha 1, JUND—JunD, TUBB—tubulin beta class 1, STC1—stanniocalcin-1, MFGE8—milk fat globule-EGF factor 8, WNT5A—Wingless-related integration site-5a.

FIG. 3A. Disruption of mechanotransduction depletes pro-fibrotic fibroblast subpopulations to prevent scar formation and allow skin regeneration. Pseudotime UMAP analysis of fibroblast transcriptional profiles using normal (no strain) fibroblasts as the point of origin.

FIG. 3B. Disruption of mechanotransduction depletes pro-fibrotic fibroblast subpopulations to prevent scar formation and allow skin regeneration. Feature plots of critical genes that contribute to myofibroblast differentiation, scar formation, or collagen degradation with corresponding violin plots to show the expression levels binned by treatment group (strain+FAKI—orange, no strain—blue, strain—green). ACTA2—actin alpha 2, smooth muscle, COL1A1—collagen type 1 alpha 1, COL3A1 collagen type 3 alpha 1, MMP1—metalloproteinase 1, MMP3—metalloproteinase 3.

FIG. 3C. Disruption of mechanotransduction depletes pro-fibrotic fibroblast subpopulations to prevent scar formation and allow skin regeneration. Pseudotime trajectory plots across the 8 Seurat clusters of these same genes.

FIG. 3D. Disruption of mechanotransduction depletes pro-fibrotic fibroblast subpopulations to prevent scar formation and allow skin regeneration. Protein level confirmation of human scRNA-seq observations using immunofluorescence staining of wounded and treated (W_HF, left) vs. wounded and untreated (W, right) porcine dermis tissue sections (from FIG. 1). Staining for alpha SMA (the protein translated from ACTA2), Collagen I (COL1A1), and Collagen III (COL3A1), MMP1 (MMP1) and MMP3 (AIMP3). Scale bar: 100 μm.

FIG. 3E. Disruption of mechanotransduction depletes pro-fibrotic fibroblast subpopulations to prevent scar formation and allow skin regeneration. Schematic showing the proposed mechanism of action demonstrating how increased mechanical stress drives fibrosis and scar formation.

FIG. 4. Timeline of porcine experiment. Full schedule of events from the time of initial injury to POD180 is shown in two separate timeline diagrams. B—biopsy, C—cutometer reading, P—photograph.

FIG. 5. Visual Analog Scale (VAS) scar scores of porcine deep partial-thickness wounds over time. Scar images collected at indicated time points (˜1, 2, and 3 months after the initial injury) were analyzed by four blinded scar experts (all board-certified plastic surgeons and wound-healing scientists with advanced degrees). Lower scores relative to each control wound indicate improvement on the five components examined as described in the Methods section (vascularity, pigmentation, acceptability, observer comfort, and contour). Statistical analysis was conducted for these plots using analysis of variance (ANOVA) with Tukey's multiple comparisons tests (**p<0.01; ***p<0.001).

FIG. 6A. Acute, systemic, and implantation toxicity testing of FAKI hydrogel in the porcine model. To assess acute toxicity, concentrated FAKI solution (150 μM and 1.5 mM dissolved in 4% DMSO and 30% PEG-300) was dripped on unwounded pig skin daily for 14 consecutive days. Wounds were monitored by gross photography, and no adverse skin reactions were observed. Blue box size=25 cm².

FIG. 6B. Acute, systemic, and implantation toxicity testing of FAKI hydrogel in the porcine model. Mass spectrometry of peripheral blood samples of FAKI-treated pigs was performed to assess systemic penetration of topically applied FAKI covering approximately 7-10% of the total body surface area relative to serum FAKI levels after oral administration of FAKI from a human study. Serum FAKI concentrations via localized delivery were negligible in three independent experiments collected from three different pigs.

FIG. 6C. The biocompatibility of FAKI hydrogels and blank hydrogels was tested by implanting a 4 cm² size hydrogel in a subcutaneous pocket closed using sutures. Tissue specimens were explanted after 14 days. Cleaved caspase-3 staining showed low levels of apoptotic cells in both conditions.

FIG. 6D. The biocompatibility of FAKI hydrogels and blank hydrogels was tested by implanting a 4 cm² size hydrogel in a subcutaneous pocket closed using sutures. Tissue specimens were explanted after 14 days. Trichrome staining showed no discernible changes in the dermal structure of overlying skin.

FIG. 7A. Fiber analysis was demonstrated using two established metrics of collagen analysis Processed images visualizing fibers for quantification of picrosirius red-stained images performed using two previously published metrics MatFiber and CT-FIRE (both in MATLAB). Analysis with both metrics demonstrated changes between treatment groups (shown in FIG. if).

FIG. 7B. Fiber analysis was demonstrated using two established metrics of collagen analysis. CT-FIRE quantification fiber count metric shows a trend in ability of FAKI treatment to return number of fibers closer to unwounded skin. Statistical comparisons were made using analysis of variance (ANOVA) with Tukey's multiple comparisons tests (*p<0.05).

FIG. 8. Regenerated intradermal adipocytes that surround secondary dermal structures are Perilipin A-positive, fully differentiated adipocytes. FAKI-treated wounds (W_HF, bottom row) at POD 90 showed regeneration of Perilipin A-positive intradermal adipocytes (red, yellow dotted arrow) in the deep dermal layer adjacent to developing appendage structures (white arrow show). There were no intradermal adipocytes in the untreated control (UW, not shown) and placebo (hydrogel only, W_H, top row) treated wounds.

FIG. 9A. 3D collagen scaffold system recapitulates observations sees in the porcine tissue. Quantification of stretch and strain of 3D stretch culture system that underwent no strain (blue, left), 10% equibiaxial strain (green, middle), or 10% equibiaxial strain+FAKI (orange, right) demonstrates effective induction of strain. Statistical comparisons were made by using analysis of variance (ANOVA) with Tukey's multiple comparisons tests (*p<0.05; ****p<0.0001).

FIG. 9B. 3D collagen scaffold system recapitulates observations sees in the porcine tissue. Alpha smooth muscle actin (alpha SMA) myofibroblast protein expression in fibroblasts cultured in all 3 conditions was quantified by immunofluorescence staining. DAPI=blue, alpha SMA=red, Scale bar: 140 μm (c) Alignment of fibroblasts within 3D stretch culture system that underwent uniaxial restraint (no strain) (blue, left), 10% uniaxial strain only (green, middle), or 10% uniaxial strain+FAKI (orange, right) was quantified using a previously published algorithm to analyze fibroblasts immunostained for phalloidin (green). Scale bar: 140 μm. Statistical comparisons were made by using analysis of variance (ANOVA) with Tukey's multiple comparisons tests (*p<0.05; ****p<0.0001).

FIG. 9C. 3D collagen scaffold system recapitulates observations sees in the porcine tissue. Contraction in vitro assay using collagen scaffolds demonstrates that disruption of mechanotransduction with FAKI hinders the fibroblasts from remodeling (R) the ECM environment. Statistical comparisons were made by using analysis of variance (ANOVA) with Tukey's multiple comparisons tests (*p<0.05; ****p<0.0001).

FIG. 9D. 3D collagen scaffold system recapitulates observations sees in the porcine tissue. Statistical comparisons were made by using analysis of variance (ANOVA) from unpaired two-tailed t-tests (#p<0.05).

FIG. 10A. Seurat mapping of fibroblast expression according to the human donor shows that fibroblasts cluster according to both strain and treatment groups, as opposed to human origin. Fibroblasts from three patients (patient 1, 2, and 3, shown in FIG. 2) do not cluster according to patient origin and instead clustered according to experimental group (no strain [NS] vs. strain [S] vs. strain+FAKI, as shown in FIG. 2.

FIG. 10B. Seurat mapping of fibroblast expression according to the human donor shows that fibroblasts cluster according to both strain and treatment groups, as opposed to human origin. Detailed look at all 9 groups between 3 human donors and the 3 treatment groups.

FIG. 10C. Seurat mapping of fibroblast expression according to the human donor shows that fibroblasts cluster according to both strain and treatment groups, as opposed to human origin. Cellular transcription profiles according to ENCODE gene database confirms that all cells within the 3D collagen system were human fibroblasts.

FIG. 10D. Seurat mapping of fibroblast expression according to the human donor shows that fibroblasts cluster according to both strain and treatment groups, as opposed to human origin. Gene enrichment pathway analysis using the KEGG GO database of genes significantly upregulated in the 8 Seurat clusters in FIG. 2B highlights the key mechanotransduction and inflammatory pathways upregulated in each cluster.

FIG. 11A. Additional gene expression profiles further demonstrate that strain increases classical pro-fibrotic and mechanotransduction markers. Pseudotime trajectories overlaid on Seurat clusters, using the normal fibroblasts as the point of origin.

FIG. 11B. Additional gene expression profiles further demonstrate that strain increases classical pro-fibrotic and mechanotransduction markers. Feature and violin plots of genes significantly upregulated with strain (left to right): yes—associated protein 1 (YAP1), Phosphoinositide-3-kinase regulatory subunit 1 (PIK3R1), Zinc Finger E-Box Binding Homeobox 2 (ZEB2), platelet-derived growth factor receptor-alpha (PDGFRA), epidermal growth factor (EGFR), mitogen-activated protein kinase 1 (MAPK1), runt-related transcription factor 1 (RUNX1), Ras-related C3 botulinum toxin substrate 1 (RAC1), and Rho Associated Coiled-Coil Containing Protein Kinase 1 (ROCK1).

FIG. 11C. Additional gene expression profiles further demonstrate that strain increases classical pro-fibrotic and mechanotransduction markers. The feature and corresponding violin plot of Mmp10—a gene significantly upregulated with FAKI treatment.

FIG. 11D. Additional gene expression profiles further demonstrate that strain increases classical pro-fibrotic and mechanotransduction markers. Pseudotime UMAP plot using S+FAKI fibroblasts as the origin point, used to create gene trajectories in FIG. 3C.

### DETAILED DESCRIPTION

As referred to herein, a large mammal is a mammal having an adult weight of greater than about 7 kg; about 10 kg; about 20 kg; about 50 kg; about 60 kg; about 70 kg, about 80 kg; about 90 kg; about 100 kg, about 150 kg; about 200 kg; about 250 kg or more. A large mammal may have a birth weight of about 0.5 kg; about 1 kg; about 5 kg; about 7 kg or more. A large mammal includes but is not limited to a cat, a dog, a human, a pig, a horse, a camel or an elephant.

As referred to herein, a focal adhesion kinase (FAK) inhibitor is a small organic molecule or biomolecule capable of inhibiting FAK, also known as PTK2, which is a mediator of signal transduction downstream of integrins and growth factor receptors in cells, including epithelial cells. While VS-6062 is described herein for use, the methods and kits are not so limited, and any suitable FAK inhibitor may be used. Some exemplary FAK inhibitors include VS-6062, PF-562271, PR-573228, TAE226 (NVP-TAE226), PF-03814735, PF-562271 HCl, GSK2256098, PF-431396, PND-1186 (VS-4718), Defactinib (VS-6063, PF-04554878), and Solanesol (Nonaisoprenol).

Tissue repair and healing remain among the most complicated processes that occur during post-natal life. After injury, humans and other large organisms heal by forming fibrotic scar tissue, which has diminished function. In contrast, smaller organisms such as planaria, salamanders, and mice respond to injury through scarless tissue regeneration with restoration of tissue function. Well established scaling principles have shown that as organisms become larger, movement requires exponentially increased peak forces within tissue. Evolution has guided compensation to these requirements by increasing organ-level mechanical properties, as seen in tissue hypertrophy and hyperplasia. However, these biologic adaptations may have unintended consequences during injury, where otherwise well-balanced forces now result in tissue fibrosis and scar formation. Applicant has discovered that blocking the biologic sensors of force in a large animal model significantly accelerates wound healing and enables tissue regeneration with recovery of secondary structures such as hair follicles. In human tissue, Applicant has demonstrated that increases in mechanical force induce a shift of fibroblast populations toward a pro-fibrotic phenotype, which is reversable with early pharmacologic blockade of force transduction signaling. For the first time, it has been shown that the fundamental relationship between biologic mass and force drives large organism fibrosis and that interrupting the native mechanisms of force transduction results in tissue regeneration, a finding that has implications for efforts to regenerate limbs, hearts, and other tissues.

A key feature that distinguishes “model organisms” from humans and other large mammals is mass, with large organisms typically several orders of magnitude larger (e.g., humans have more than 10⁵-fold the mass of planaria). While evolution has allowed model organisms the ability to fully regenerate in environments of low mechanical stress, the ability to withstand increased tissue forces has allowed mammals to grow larger in mass and increase in biologic complexity. Well established scaling principles dictate that as organisms evolve and grow larger, peak stresses within their tissues increase exponentially during locomotion and movement. Evolution has shaped the development of large organisms to compensate for these increased forces in a variety of ways, from fundamental changes such as tissue hypertrophy to more complex adaptations as seen in the alteration of limb posture to reduce forces experienced by bone and muscle during locomotion. Other organs with the inability to relieve these forces, such as skin, are compelled instead to adapt by altering and increasing mechanical properties to handle these forces. These scaling principles governing the relationship between biologic mass and force explain the development of fibrosis within humans.

Various recent efforts have studied regenerating model organisms in order to pinpoint genes, proteins, or signaling pathways that could be utilized to promote regeneration in humans. However, while novel target pathways may have been identified, it has been unknown as to the effect that increasing mechanical forces may have on these processes.

Applicant has surprisingly discovered that mechanical stress in tissue in large organism regeneration is critically important. High mechanical stress encourages fibrogenic phenotypes and collagen deposition by fibroblasts, leading to fibrotic scar formation that can critically interfere with regenerative processes. Blocking mechanotransduction can suppress the exuberant collagen deposition and fibrotic characteristics of physiologic wound healing in large organisms, and thus may lead to accelerated, scarless wound healing and subsequent skin regeneration. By inhibiting cells from sensing the physiologically high levels of mechanical stress, pro-fibrotic subpopulations may be eliminated and reduce or eliminate consequent scar formation. This may permit other cells to migrate into the wound to restore normal skin composition and thereby generate tissue regeneration in large organisms. Applicant has found that mechanical stress and cellular mechanotransduction signaling pathways are important factors to be considered when attempting to achieve true regeneration in large mammals and human patients. Applicant is the first to discover that disrupting the fundamental relationships between mass and force can erase the evolutionary tradeoff between organism complexity and regenerative capability, driving large organisms toward “model organism”-like tissue regeneration. In particular, Applicant has discovered that disrupting focal adhesion kinase (FAK), a key biological sensor of force, may enable tissue regeneration following injury in humans and other large mammals, and may provide profound implications for efforts to regenerate limbs, hearts, and other tissues.

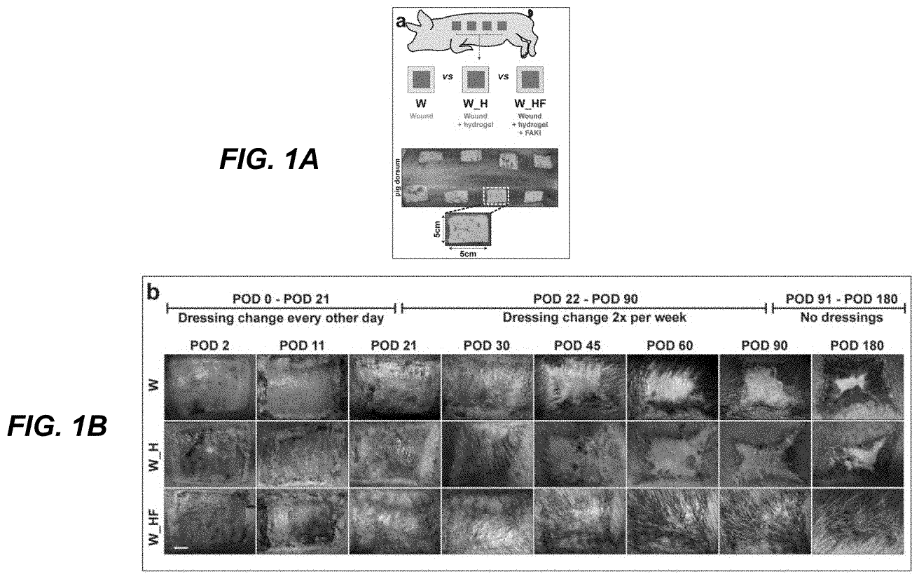

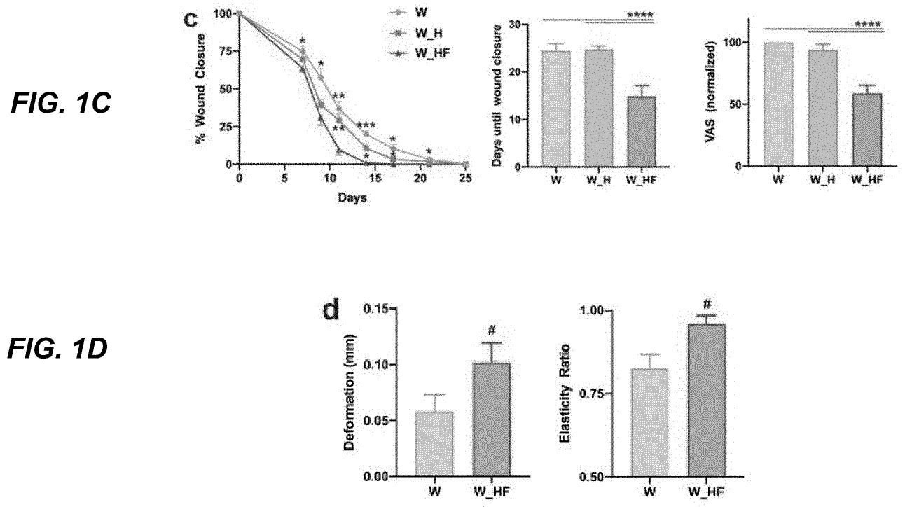

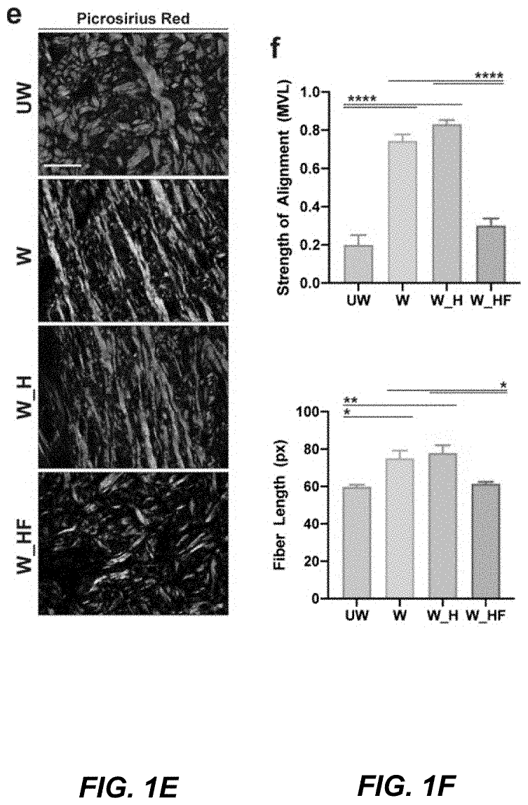

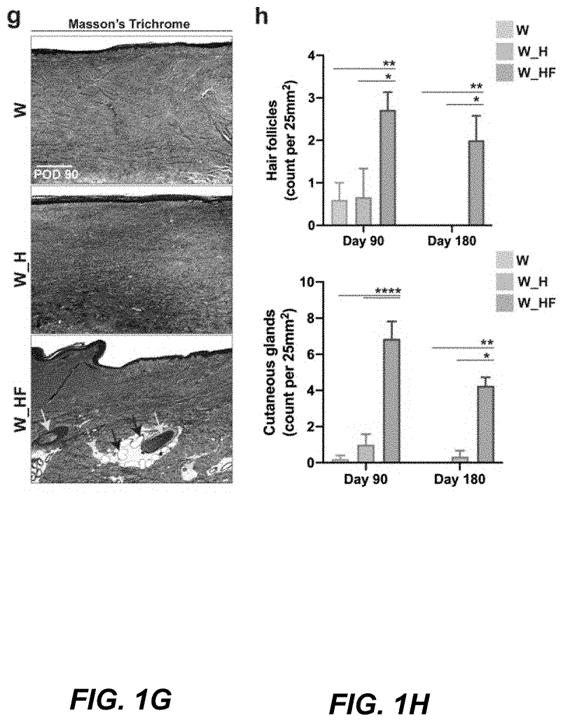

Inhibiting biological sensors of force in large animal wounds allows for tissue regeneration with minimal fibrotic response. FAK signaling has been identified as an upstream mediator for transferring tissue-level integrin-matrix force sensory interactions to downstream cellular pathways. To evaluate the effects of blocking mechanotransduction on tissue repair in large animals, a pharmacologic inhibitor of FAK (FAK-I, VS-6062) was explored. This compound was previously demonstrated to have effectiveness as an anti-cancer therapy to treat advanced solid tumors in clinical trials. Excisional wounding in the red Duroc pig was selected as the model organism being a large animal widely considered the most similar to humans in terms of skin physiology and cutaneous wound healing. (See FIG. 1A). Both humans and the red Duroc heal from deep dermal injuries by developing thick, collagenous hypertrophic scars (HTS) that replace the physiologic soft skin tissue. Compared to unwounded skin, this scar tissue never has hair follicle or skin appendage recovery. Instead, this tissue is characterized by a thickened dermis and absence of intradermal fat, resulting in increased mechanical stiffness.

Disruption of mechanotransduction was explored using a small molecule FAK inhibitor (FAKI) to determine whether wound healing kinetics could be modulated. As described in the Experimental section, wounds treated with FAKI, using a sustained release hydrogel scaffold, were found to be fully healed at postoperative day (POD) 14±2.3, more than 10 days earlier than wounds treated with standard dressings or empty hydrogels, which both healed after POD 24, as shown in FIGS. 1B-1C. Furthermore, pharmacologic blockade of mechanical signaling resulted in normal appearing skin in treated pig wounds as shown in FIG. 1C. This noticeable difference in scarring was evident as early as POD 40 (See FIG. 5). Using a tissue cutometer, a non-invasive clinical instrument that measures the biomechanical properties of skin, wounds treated with FAKI exhibited tissue properties similar to that of unwounded skin, including decreased firmness and increased elasticity, as shown in the bar graph of FIG. 1D. Taken together, disrupting the ability of cells to sense tissue mechanical stress following cutaneous wounding may lead to accelerated healing and the recovery of normal skin characteristics.

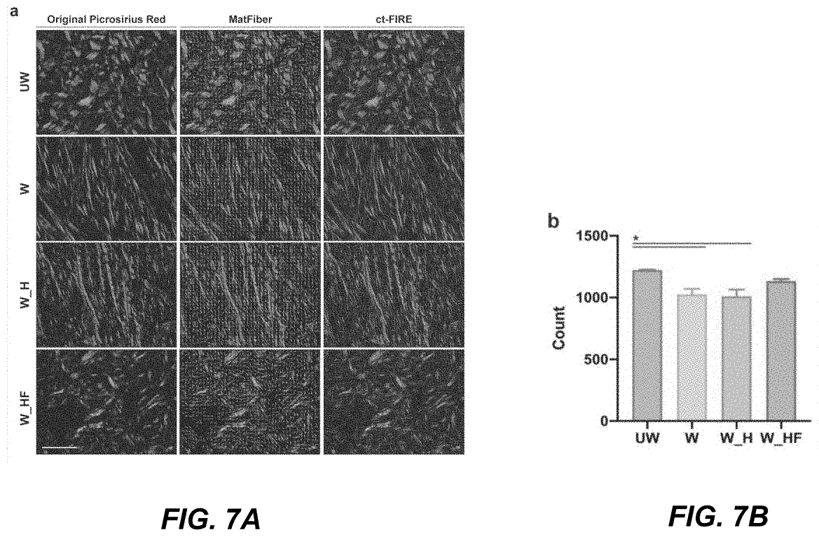

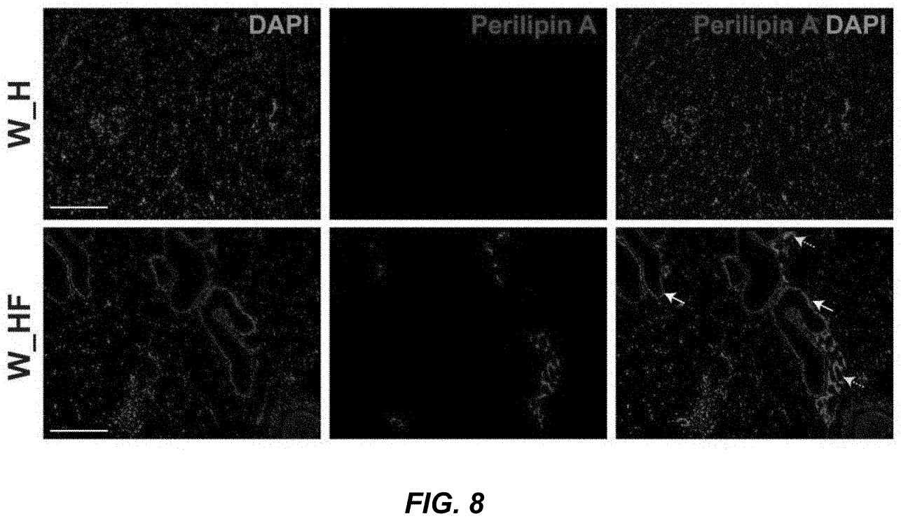

Quantitative assessment of tissue ultrastructure in untreated pig wounds revealed significant fibrosis, demonstrated by collagen elongation and increased unidirectional fiber alignment, as shown in FIG. 1E. Wounds treated with pharmacologic blockade of mechanotransduction, by contrast, healed with the basket weave-like collagen structure characteristic to unwounded porcine skin, as shown in FIGS. 1E-1F and FIGS. 7A-7B. Furthermore, it was discovered that these treated wounds exhibited regrowth of hair follicles and other cutaneous glands, similar to native skin, whereas wounds allowed to experience mechanical force without treatment did not achieve regeneration of secondary structures, as shown in FIGS. 1G-1H and FIG. 8). Without being bound by theory, these results indicate that mechanical signaling may be a critical mediator of fibrotic tissue development during wound healing. Disrupting this signaling may reduce fibrosis, restore normal skin architecture, and promote secondary structure regeneration.

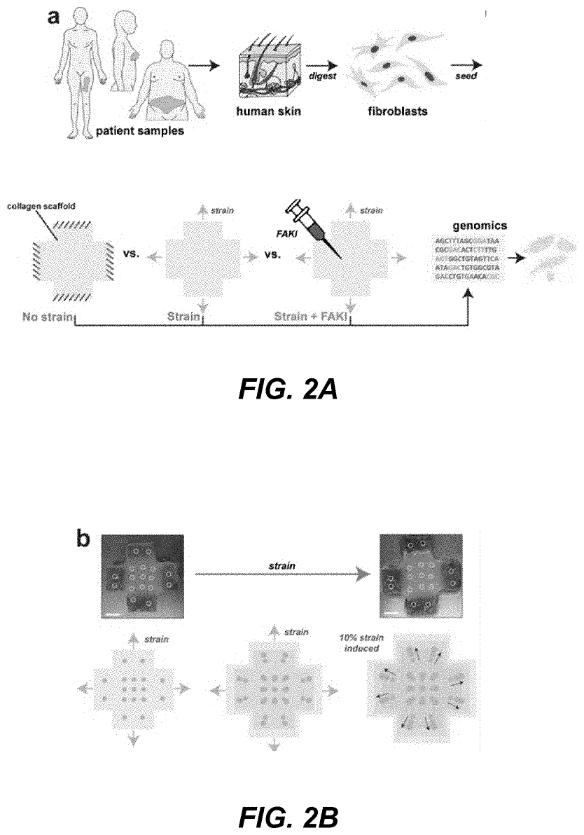

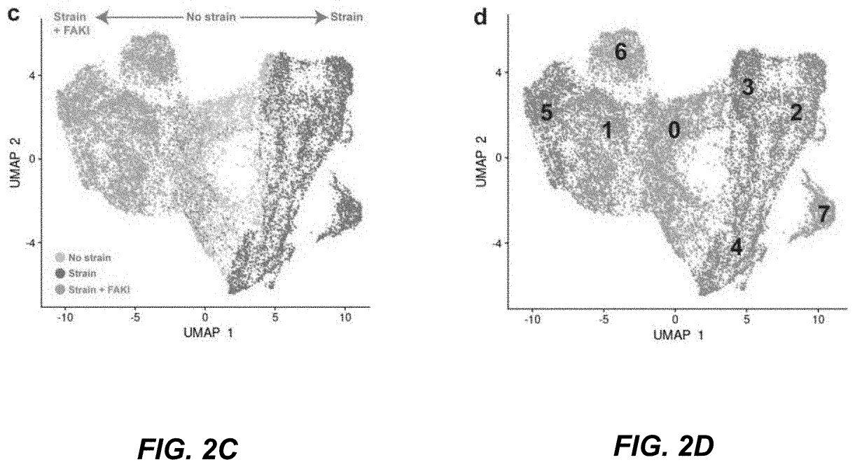

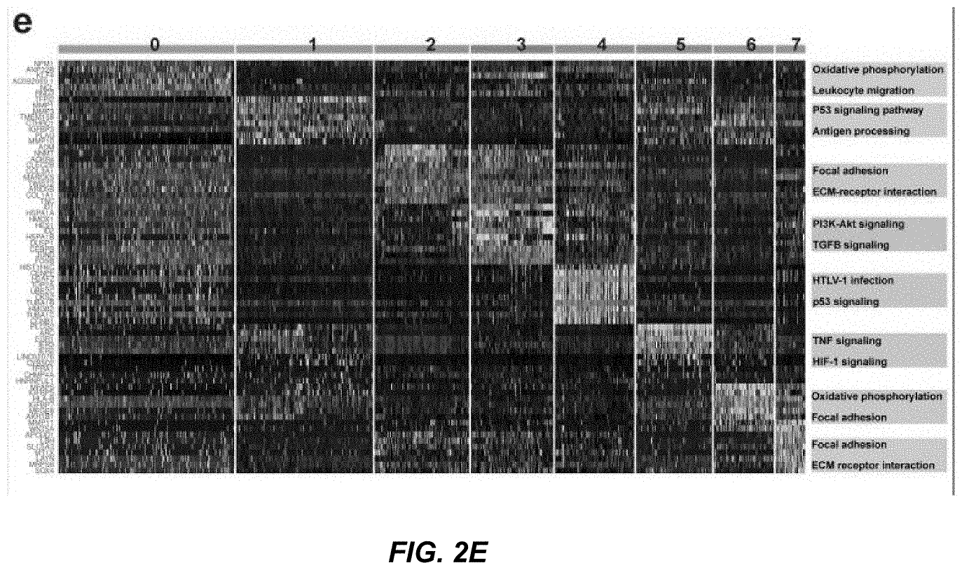

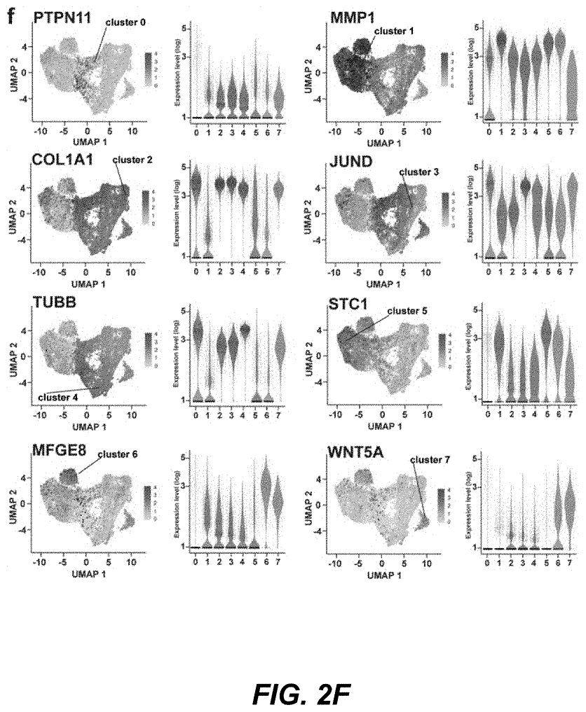

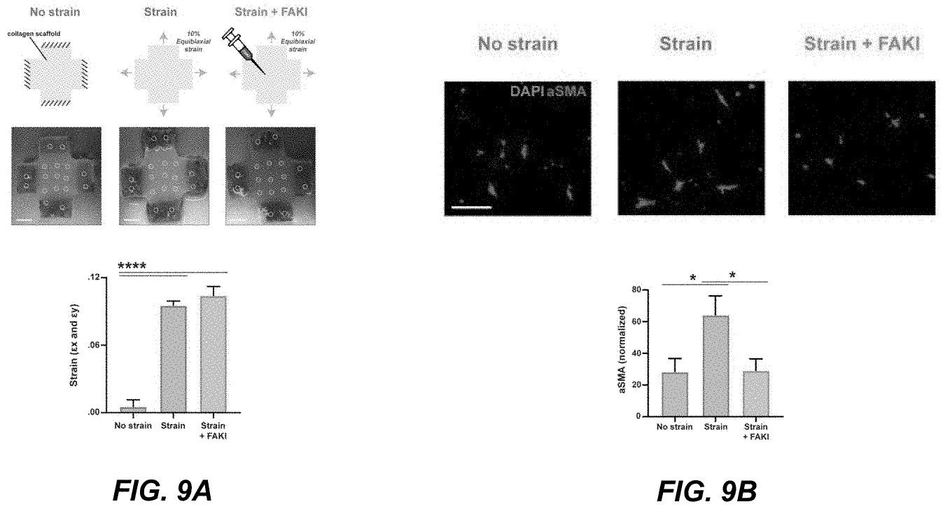

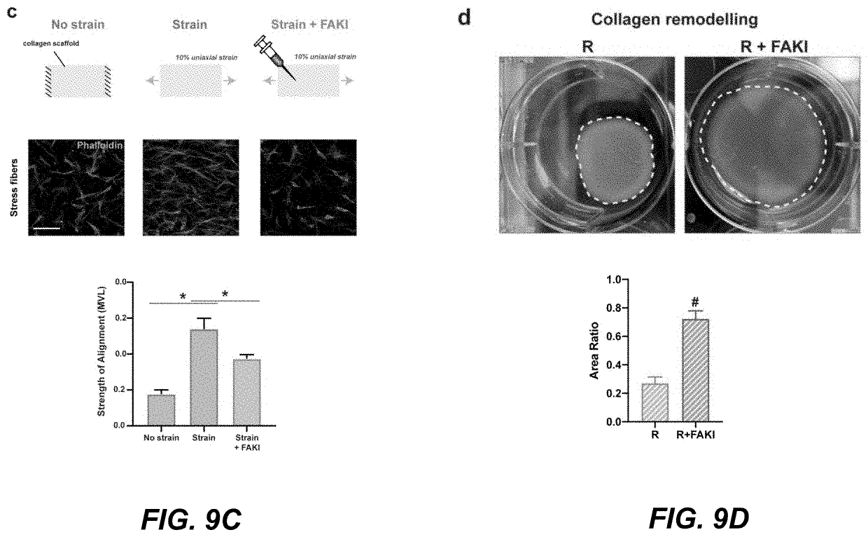

Mechanotransduction shifts human fibroblast heterogeneity. Evolutionary pressures to increasing mass in large organisms have resulted in organs developing with increased durability and mechanical properties. To confirm the translational potential of the results in the porcine model on the role of mechanotransduction in the fibrotic response, dermal fibroblasts isolated from human surgical patients were then evaluated. Cells were seeded within 3D collagen scaffolds at densities of 2.0 mg/mL collagen and 200,000 cells/mL (FIG. 2A). To artificially increase tissue forces, the mechanical strain applied to fibroblasts was precisely manipulated as described in Chen, K. et al, “Role of boundary conditions in determining cell alignment in response to stretch”, PNAS 115, 986-991, doi:10.1073/pnas.1715059115 (2018), the entire disclosure of which is hereby incorporated by reference in its entirety, and shown in FIG. 2B and FIG. 9A. In a subset of cells, mechanotransduction signaling with FAKI treatment was also blocked immediately after application of strain. The efficacy of this in vitro system to mimic the fibrogenic phenotype observed in healing wounds and resultant scar was confirmed. Specifically, dermal fibroblasts cultured in a uniaxial strain environment demonstrated uni-directional, elongated cellular alignment, as shown in FIG. 9C. In contrast, fibroblasts blocked from sensing mechanical forces aligned multi-directionally, demonstrating a restoration in alignment to normal architecture, which was similar to that seen in porcine skin, as shown in FIGS. 1E-1F. Fibroblast reorganization of ECM drives collagen remodeling and development of long, aligned collagen fibers characteristic of fibrosis. Consequently, inhibition of mechanotransduction reduced the fibroblasts' ability to reorganize collagen and remodel their surrounding environment, as shown in FIG. 9D. This 3D collagen scaffold system recapitulates both the fibrogenic responses observed during normal wound healing and the ability to block those responses.

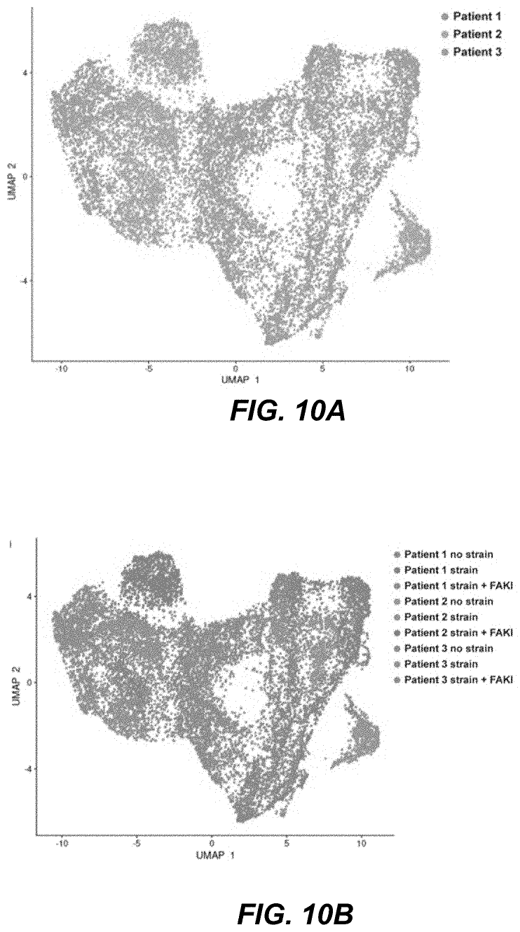

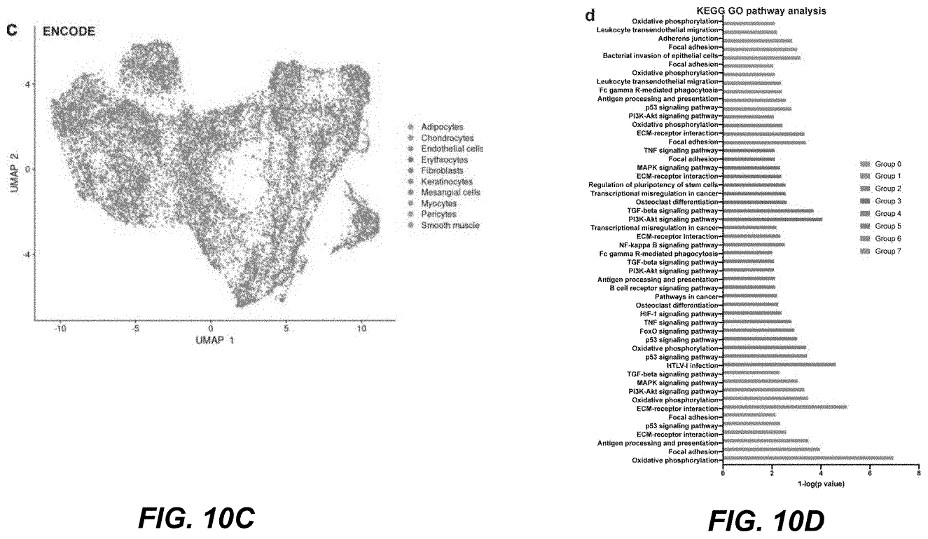

Using this experimental system, in vivo dermal strain patterns were mimicked, imposing biaxial strain upon the fibroblasts, while also attenuating mechanotransduction in a subset of samples. The collagen scaffolds were enzymatically digested to obtain cellular suspensions of fibroblasts, which were then processed for scRNA-seq, as shown in FIGS. 2A and 2C. All single cell data were initially analyzed in a manner blinded to phenotype of origin. Following log-normalization, pooled data were subjected to semi-supervised Louvain-based clustering and embedded into UMAP-space (Seurat). Eight transcriptionally distinct subpopulations (cluster 0 to cluster 7) were identified among cells from the pooled data superset, as shown in FIG. 2D. When unblinded, considerable permissiveness among clusters were found across both phenotypes and biological replicates, consistent with the known heterogeneity among human dermal fibroblasts (FIG. 2D). Automated cell-level annotations were obtained using the SingleR toolkit against the ENCODE Blue database, supporting that all cells used in the experiments displayed fibroblast transcriptional programs (FIG. 10C).

Unstrained fibroblasts were found to aggregate together as a relatively homogeneous group near the center of UMAP embedding, representing the overwhelming majority of cells in the putative cluster 0. These cells, defined primarily by consistent expression of fibroblast genes such as PTPN11 and HADHA, a well established housekeeping gene upregulated in cluster 0, likely represent the native fibroblast steady-state in the experimental system (FIGS. 2C-2D) By contrast, fibroblasts subjected to mechanical strain immediately prior to sequencing were found to have considerably altered transcriptomic profiles and a comparatively heterogeneous dispersion within the data manifold. These strained fibroblasts were distributed primarily among clusters 2, 3, 4, and 7 and were defined by differential expression of pro-fibrotic genes such as COL1A1, COL3A1, JUND, TUBB, and WNT5A (FIGS. 2C-2D).

Finally, when mechanotransduction signaling in fibroblasts was pharmacologically disrupted immediately prior to application of strain, the resulting transcriptional programs were shifted toward a new meta-state in the opposite ‘direction’ from cells that were strained without treatment. These treated cells were mapped almost exclusively to the putative clusters 1, 5, and 6, and defined by differential over-expression of genes known to drive ECM degradation, such as IMP1 and AIP3, as well as anti-fibrotic genes such as STC1 and MFGE8. A small subset of treated fibroblasts even appeared to revert back to a transcriptional programming consistent with unstrained fibroblasts—something that was not observed in any cells from strained and untreated fibroblasts. Furthermore, the global shifts in fibroblast transcriptional signatures among treated cells were robust and preserved across three diverse human samples, each collected from different anatomical locations from different patients (FIG. 2C; FIGS. 10A-10B). Modulation of mechanotransduction can “push” and “pull” fibroblast programming either toward or away from a fibrotic transcriptional state.

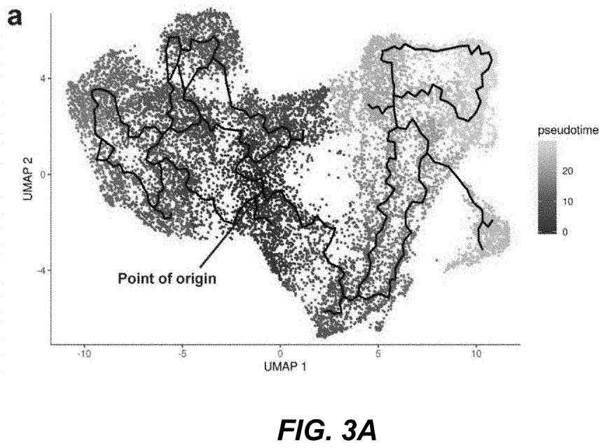

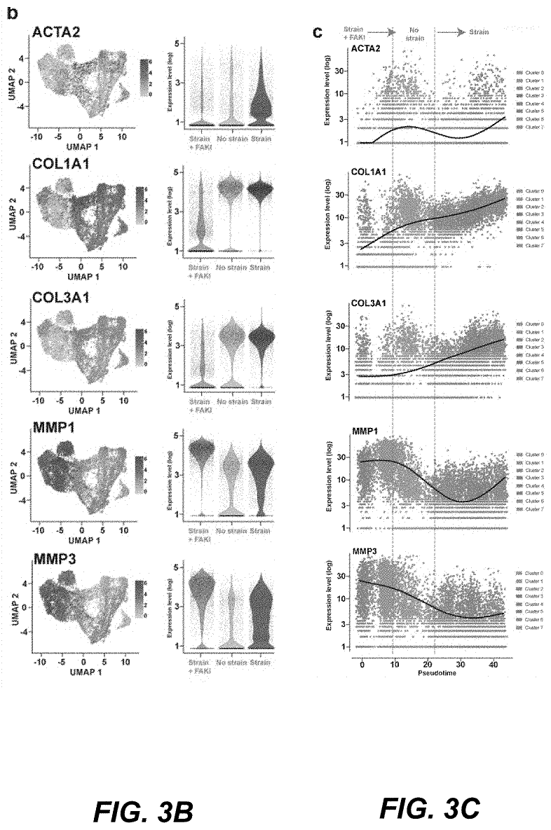

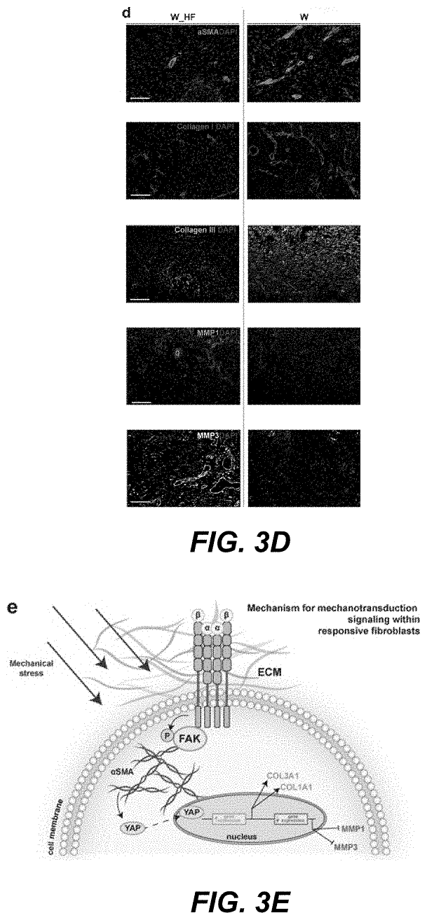

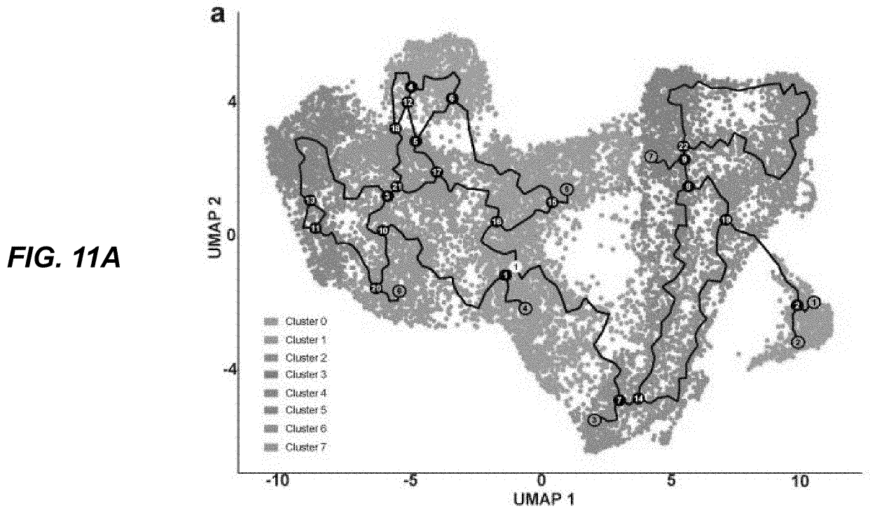

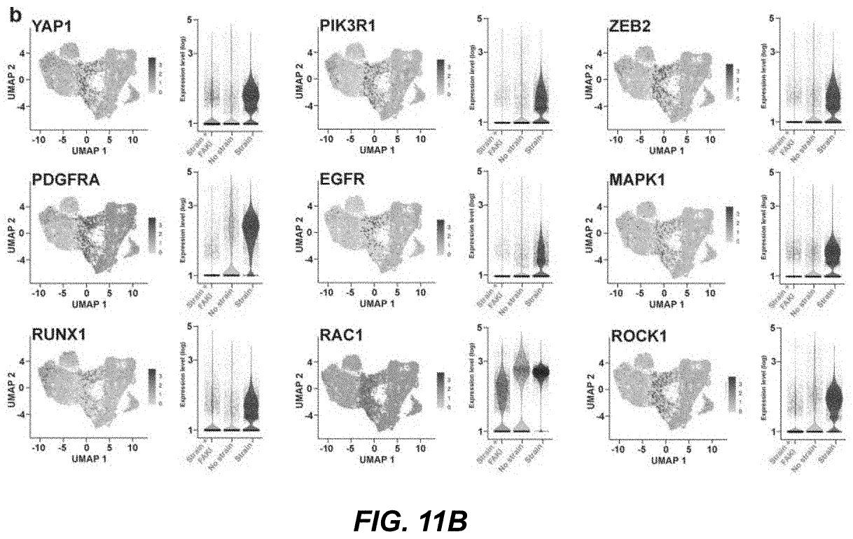

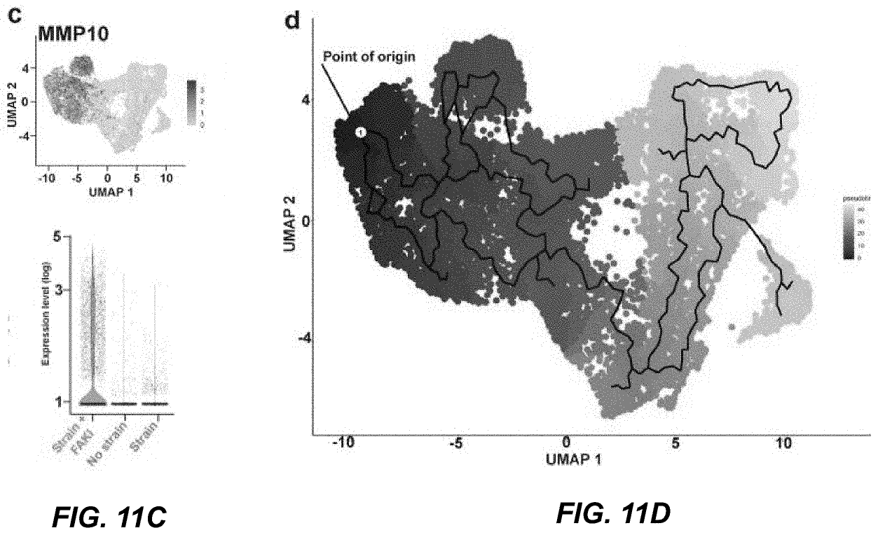

Disruption of mechanotransduction prevents the enrichment of force-responsive pro-fibrotic subpopulations and globally shifts fibroblasts toward a stress-shielded putatively-regenerative state. To further investigate the transcriptional shifts observed in the single cell data, pseudotime trajectories were constructed based on phenotypic state. Defining unstrained fibroblasts as the point of origin, it was found that mechanically strained fibroblasts showed markedly stronger transcriptional differences along the associated pseudotime trajectories compared to strained fibroblasts that were also treated with FAKI (FIG. 3A; FIG. 11A). This suggests that treatment of fibroblasts during strain not only alters the resulting program, but does so in a way that less transcriptionally distinct than their strained fibroblasts without treatment. [0045] Further analysis of transcriptomic signatures showed that mechanically strained fibroblasts exhibited greater expression of classical pro-fibrotic markers (ACTA2, PDGFRA), markers promoting myofibroblast differentiation (RUNX1, ZEB2), and downstream mechanotransduction signaling pathway genes (MAPK1, PI3KR1, EGFR, RAC1, YAP1) (FIG. 3B; FIG. 11B). Suppression of mechanotransduction within these strained fibroblasts abrogated nearly all of these fibrotic signaling changes (FIG. 3B; FIGS. 11B-11C).

Examining the downstream products of fibrosis, although both unstrained and strained fibroblasts demonstrated high expression of COL1A1 and COL3A1 mRNA, FAKI treatment strongly reduced the transcription of these ECM component genes. This pharmacological blockage also increased expression of MMP1 and AIMP3, key enzymes involved in the degradation of collagen and known to reduce fibrosis across a wide range of disease models. To further demonstrate these findings, the mechanically disrupted fibroblasts were set as the point of origin in pseudotime to map the progression of cellular transcriptional signatures from mechanically disrupted to normal and finally to transcriptionally distinct, strained fibroblasts (FIG. 3C; FIG. 11D). By ordering the pseudotime trajectory in this order, an “axis of regeneration” was demonstrated that inversely correlates to increasing mechanical stress. Along this axis, fibrotic genes increase while putative-regenerative genes decrease.

To confirm at the protein level, the human fibroblast findings were applied to tissue blocks from the large animal comparator. Immunofluorescent staining was performed on wounded porcine tissue at specific time points, staining tissue collected from the pig wounds as shown in FIGS. 1A-1B for the images shown in FIG. 3D (as described above in paragraphs [0035] to [0037]). Protein expression of alpha SMA (the translational product of ACTA2), Collagen I (COL1A1), and Collagen III (COL3A1) was decreased in FAKI treated wounds, while MMP1 (MMP1) and MMP3 (AIMP3) increased. Thus, changes in protein expression within the porcine scar lesions were consistent with gene expression data determined by scRNAseq analysis of human fibroblasts (FIGS. 3B-3D). Normal porcine wounds displayed increased myofibroblast differentiation, demonstrated by increased alpha SMA expression which in turn resulted in increased collagen deposition. In contrast, wounds with disrupted mechanical signaling demonstrated decreased myofibroblast differentiation and collagen production, along with increased MMP1 and MMP3 expression. These data corroborate the findings in human cells, establishing the critical role of mechanical signaling in wound healing and scar formation in large organisms.

These experiments were designed to test the hypothesis that increases in mechanical stress would directly lead to the pro-fibrotic phenotype seen during physiologic wound healing in humans. As organisms have grown larger in size, they have biologically adapted by increasing the mechanical strength of their tissue. Without being bound by theory, these increased mechanical forces present during the healing process may promote scar formation and prevent true regeneration through the following mechanism (FIG. 3E). Increased mechanical stress may trigger activation of integrins and FAK, which in turn may promote alpha SMA expression and subsequent myofibroblast differentiation. Alpha SMA stabilization may promote expression of transnuclear proteins, such as through the YAP-TAZ pathway, which translocate into the nucleus to promote a cascade of pro-fibrotic signals, demonstrated by increased mechanotransduction signaling and COL1A1 and COL3A1 expression, leading to exuberant collagen deposition and fibrotic scar formation. Disruption of mechanotransduction, however, may inhibit these signal cascades from occurring and eliminate these pro-fibrotic fibroblast subpopulations, while also promoting expression of enzymes that reduce scar tissue, such as MMP1 and MMP3. MMPs such as MMP1 degrade not only existing scar tissue but also a variety of provisional matrix proteins that make up the acute wound bed after injury. Further, MMPs may also promote cellular migration into the wound and re-epithelialization by surrounding keratinocytes, leading to accelerated healing. By preventing scar formation and promoting MMP expression, regenerative cell subpopulations may be induced to migrate into the wound and promote skin regeneration.

Kits. Kits are provided for promoting tissue healing while reducing fibrosis in a large mammal. A kit may include a composition, where the composition includes a FAK inhibitor configured for local administration to a wounded tissue of the large mammal. In some embodiments, the composition may include a porous scaffold and the FAK inhibitor is disposed in pores of the porous scaffold. In some variations, the porous scaffold may include a hydrogel. In some variations, the porous scaffold hydrogel may be a thin film. In some embodiments, the hydrogel may be a pullulan-collagen hydrogel. In some variations, the kit further may include a wound dressing configured to protect the wounded tissue.

### EXPERIMENTAL

FAKI-releasing pullulan-collagen hydrogel production: All laboratory procedures for FAKI-releasing hydrogel patch production were conducted as described in Ma et al., “Controlled Delivery of a Focal Adhesion Kinase Inhibitor Results in Accelerated Wound Closure with Decreased Scar Formation”, J. Invest Dermatology (2018) 138, 2452-2460, the disclosure of which is incorporated by reference in its entirety. FAKI (VS-6062) compound was obtained from Verastem Oncology (Needham, Mass.) and Selleckchem (Houston, Tex.). Packaged FAKI hydrogel patches in their final form were sterilized with e-beam irradiation by a third-party company (Steri-Tek, Fremont, Calif.), and maintained in an air-tight package until use.

Animal Care: All animal work was conducted in accordance with the Administrative Panel on Laboratory Animal Care (APLAC #31530 and 32962) protocol approved by Stanford University. Seven female red Duroc pigs, 6-8 weeks old and weighing approximately 16-20 kg at the time of surgery, were purchased from Pork Power Farms (Turlock, Calif.). All animals were acclimated for at least one week upon arrival. All animals were fed lab porcine grower diet and water ad lib.

Porcine deep partial-thickness excisional wound model: Prior to operation, animals were administered oral amoxicillin 10 mg/kg for 24 hours. General anesthesia was administered by Veterinary Services personnel and was established with intramuscular telazol 6-8 mg/kg, administered once as a pre-anesthetic. Animals were then intubated using an endotracheal tube and maintained on 1.5-3% of inhaled isoflurane throughout the procedure. The hair on the back was clipped and skin was cleansed initially with Betadine© solution following by a 70% alcohol rinse. Excisional wounds were created with a standard electric Zimmer dermatome (Zimmer Biomet, Warsaw, Ind.). Up to eight wounds, approximately 5 cm×5 cm in size, were created on each lateral flank, with 3-5 cm intervals between wounds (FIG. 1A). Multiple dermatome passes were performed to create deep partial-thickness wounds of uniform 0.07 inch depth. The wounds were randomly assigned to receive either FAKI hydrogel (W_IF), blank hydrogel (‘placebo’, W_H), or no hydrogel (wounded control, W) (n=6-9 wounds per condition). Animals were given oral amoxicillin 10 mg/kg post-operatively twice a day for 5 days total. Wound dressings, including FAKI hydrogel patches, were changed every other day for the first three weeks after initial injury until POD 21 (FIG. 1C). Thereafter, dressings were changed twice per week until POD 90. Animals were subject to short-term sedation for each dressing change. The actual dose delivered to the wound was tested on porcine fresh wounds (red Duroc pigs). In a time-dependent study, the amount of VS-6062 detected at approximately 0.5 mm wound depth over 24 hours ranged from 30-100 microG/g tissue by weight. The amount of VS-6062 detected beyond 1 mm wound depth was less than 5 microG/g tissue.

Wound closure, visual scar assessment, and viscoelastic analyses. Wounds were monitored photographically at each dressing change. Days to wound closure, defined as complete re-epithelialization without open wound area, were determined for each wound based on gross photographic assessment. Quantification of scar metrics were performed using a Visual Analog Scale (VAS) for 5 components (vascularity, pigmentation, observer comfort, acceptability, and contour) by a panel of four blinded scar experts. Total scores are calculated as a composite of all 5 scores; lower scores indicate improved scar appearance. A Cutometer (Dual MPA 580, Courage+Khazaka Electronic, Köln, Germany) was used to evaluate the firmness and elasticity of the healing wounds at POD 60. The cutometer measures the vertical deformation of the skin surface by applying a negative pressure (suction) through a small circular diameter (2 mm probe). Cutometer assessment is the gold standard to measure viscoelasticity in human patients. Deformation (suction) for two seconds followed by two seconds of relaxation (no suction) is applied for three cycles. The elasticity ratio (ability for tissue to return back to original setpoint) was measured during the relaxation period (R2 metric).

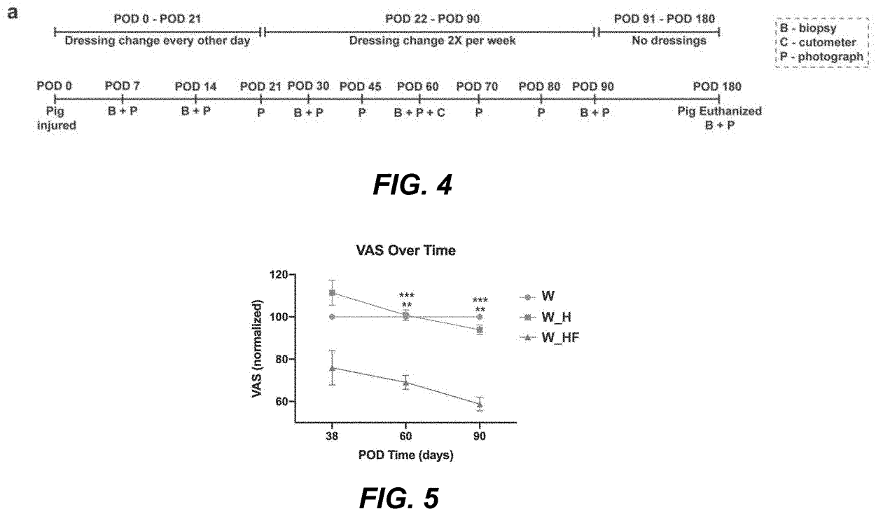

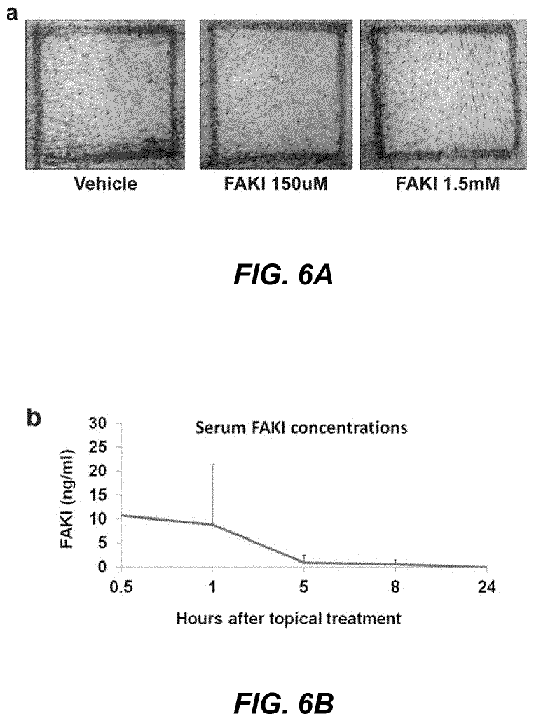

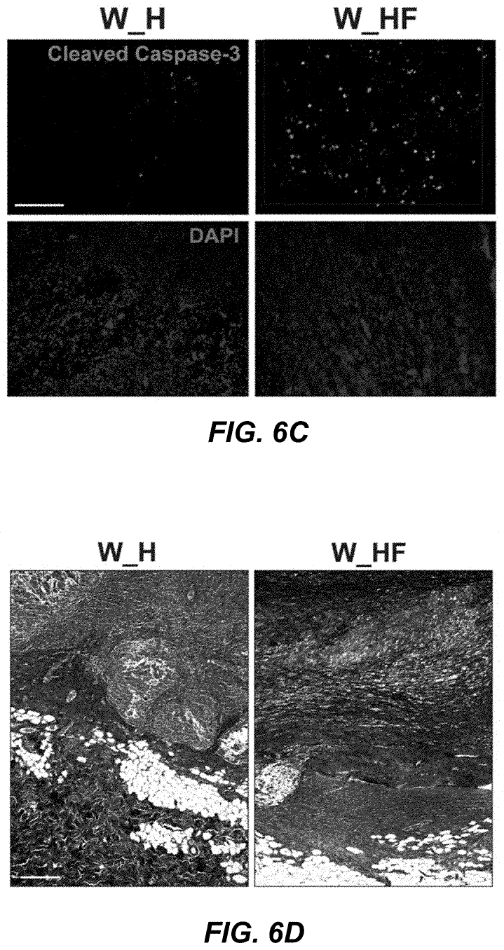

Histological and immunofluorescent staining. Specimens were harvested from the center of each wound at intermediate time points and at the end of the study, as shown in FIG. 4, fixed in 4% paraformaldehyde, dehydrated, and then paraffin embedded. Masson's Trichrome staining and Picrosirius Red staining were performed. Picrosirius Red-stained images were captured using polarized light microscopy (Leica DM5000 B upright microscope). Collagen fibers were also visualized using Second Harmonic Generation (SHG) on a Leica SP5 upright confocal multi-photon microscope using a 20λ objective. SHG images were captured with an excitation wavelength of 860 nm, a pulse length of approximately 100 fs, and an emission filter centered at 445 nm with a 20-nm bandwidth. Forward SHG was used to image fibroblasts and backward SHG was used to image in vivo tissue sections. Analysis of fiber alignment was performed on Picrosirius Red-stained images at 40× magnification using the custom software MatFiber, an intensity-gradient-detection algorithm for analysis of overall alignment of collagen fibers and stress fibers from multiple samples. The mean vector length (MVL) represents the strength of alignment and ranges from a value of 0 (completely random fiber alignment) to 1 (completely aligned fibers). The overall strength of alignment of the fibers were calculated. (See Chen, K. et al, “Role of boundary conditions in determining cell alignment in response to stretch”, PNAS 115, 986-991, doi:10.1073/pnas.1715059115 (2018), the entire disclosure of which is hereby incorporated by reference in its entirety.). Analysis of the total number of fibers was calculated using the Matlab source code for CT-FIRE Individual Fiber Extraction. Immunofluorescent staining was performed using primary antibodies targeting α-smooth muscle actin (alpha SMA), Collagen I, Collagen III, increased while expression of MMP1, and MMP3 were purchased from either Abcam (Burlingame, Calif.) or Cell Signaling Technology (Danvers, Mass.). The percentage of fluorescent area was quantified using a custom MATLAB image processing code (See Chen, K. et al, “Role of boundary conditions in determining cell alignment in response to stretch”, PNAS 115, 986-991, doi:10.1073/pnas.1715059115 (2018), the entire disclosure of which is hereby incorporated by reference in its entirety.) All histology and immunofluorescent images shown are representative images of multiple experiments.

Fibroblast-populated 3D collagen scaffold experiments. Dermal fibroblasts were isolated from both porcine and human skin samples and cultured separately. Porcine skin was obtained from the unwounded (normal skin) areas of a euthanized red duroc pig. Human skin samples were obtained under the approved IRB (#54225) and collected from three surgical procedures; a breast mastectomy, an abdominoplasty, and a thighplasty (n=3 patients). Fibroblasts were isolated by mechanical and enzymatic digestion and cultured under standard conditions until passage 3. The primary fibroblast cultures were then used to create fibroblast-populated collagen hydrogels at final concentration of 200 k cells/mL and 2 mg/mL collagen (PureCol, Advanced Biomatrix, San Diego, Calif.), following protocols as described in Chen, K. et al, “Role of boundary conditions in determining cell alignment in response to stretch”, PNAS 115, 986-991, doi:10.1073/pnas.1715059115 (2018), the entire disclosure of which is hereby incorporated by reference in its entirety. In brief, collagen scaffolds were formulated in a cruciform shape in petri dishes with a PDMS coating (˜4 mm) on the bottom (FIG. 2B). Pins were pushed through the hydrogel cruciform arms to constrain the scaffolds in both directions for a 24h pre-culture period before being subjected to either no strain, 10% equibiaxial strain, or strain+FAKI treatment for an additional 48 hours (FIGS. 2A-2B, FIG. 9A). FAKI treatment was administered by adding 20 mM FAKI in DMSO into the culture media of the scaffolds to achieve a final concentration of 10 micromolar FAKI for 48 hours. Strain was imposed by removing the anchoring pins, manually extending the hydrogel cruciform arms, and pushing the pins back to hold the arms in the new, extended position. Nine Titanium (IV) oxide paint dots (Sigma-Aldrich) were applied on the surface of the central region of the gel (boxes in FIG. 9A) to track and quantify the imposed strains. A digital camera was used to image the markers before and after strain. Photographs of marker position were used to compute a single homogenous deformation gradient tensor F that provided the least-squares best fit mapping of the 9 marker positions from the undeformed to deformed positions by solving the overdetermined matrix equation:

x=FX+p (1)
where p is an arbitrary vector included to account for translation between images. The deformation was converted to a strain tensor E using:

$$E = \frac{1}{2} ( {[ {F}^{T} F ]}^{2} - I ) \qquad ( 2 )$$

Single cell barcoding, library preparation, and single cell RNA sequencing. After two days of increased (induction of strain) or inhibited (induction of strain+FAKI) mechanotransduction, collagen scaffolds were micro-dissected and enzymatically digested to obtain cellular suspensions of human fibroblasts for droplet-based microfluidic single cell RNA sequencing (scRNA-seq) using the 10× Chromium Single Cell platform (FIG. 2C) (Single Cell 3′ v3, 10× Genomics, USA). A droplet of the cell suspensions, reverse transcription master mix, and partitioning oil was loaded onto a single cell chip and processed on the Chromium Controller. Reverse Transcription was performed at 53° C. for 45 min. cDNA was amplified for 12 cycles total (BioRad C1000 Touch thermocycler) with cDNA size selected using SpriSelect beads (Beckman Coulter, USA) and a ratio of SpriSelect reagent volume to sample volume of 0.6. cDNA was analyzed on an Agilent Bioanalyzer High Sensitivity DNA chip for qualitative control purposes. cDNA was fragmented using the proprietary fragmentation enzyme blend for 5 min at 32° C., followed by end repair and A-tailing at 65° C. for 30 min. cDNA were double-sided size selected using SpriSelect beads. Sequencing adaptors were ligated to the cDNA at 20° C. for 15 min. cDNA was amplified using a sample-specific index oligo as primer, followed by another round of double-sided size selection using SpriSelect beads. Final libraries were analyzed on an Agilent Bioanalyzer High Sensitivity DNA chip for qualitative control purposes. cDNA libraries were sequenced on a HiSeq 4000 Illumina platform aiming for 50,000 reads per cell.

Data processing, FASTQ generation, and read mapping. Base calls were converted to reads using the Cell Ranger (10× Genomics; version 3.1) implementation mkfastq and then aligned against the GRCh38 v3.0.0 (human) genome using Cell Ranger's count function (an implementation of STAR v2.7.0) with SC3Pv3 chemistry and 5,000 expected cells per sample⁴². Cell barcodes representative of quality cells were delineated from barcodes of apoptotic cells or background RNA based on a threshold of having at least 200 unique transcripts profiled, less than 10,000 total transcripts, and less than 10% of their transcriptome of mitochondrial origin.

Data normalization and cell subpopulation identification. Unique molecular identifiers (UMIs) from each cell barcode were retained for all downstream analysis. Raw UMI counts were normalized with a scale factor of 10,000 UMIs per cell and subsequently natural log transformed with a pseudocount of 1 using the R package Seurat (version 3.1.1). Highly variable genes were identified, and cells were scaled by regression to the fraction of mitochondrial transcripts. Aggregated data was then evaluated using uniform manifold approximation and projection (UMAP) analysis over the first 15 principal components. Cell annotations were ascribed using SingleR toolkit (version 3.11) against the ENCODE blue database.

Generation of characteristic subpopulation markers and enrichment analysis. Cell-type marker lists were generated with Seurat's native FindMarkers function with a log fold change threshold of 0.25 using the ROC test to assign predictive power to each gene. The 100 most highly ranked genes from this analysis for each cluster were used to perform gene set enrichment analysis against pathway databases in a programmatic fashion using EnrichR (version 2.1).

When a feature or element is herein referred to as being “on” another feature or element, it can be directly on the other feature or element or intervening features and/or elements may also be present. In contrast, when a feature or element is referred to as being “directly on” 15 another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/of” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the FIGS. It will be understood that the spatially relative terms are intended to encompass different orientations of the device in use or operation in addition to the orientation depicted in the FIGS. For example, if a device in the FIGS. is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A kit for promoting tissue healing while reducing fibrosis in a large mammal, comprising a composition comprising a FAK inhibitor disposed in pores of a pullulan-collagen hydrogel scaffold configured for local administration to a wounded tissue of the large mammal and instructions for administering, the composition, wherein the large mammal has an adult weight of greater than about 7 kilograms, wherein the composition comprising the FAK inhibitor is formulated to deliver at a concentration of about 30 to about 100 micrograms/g tissue by weight of the FAK inhibitor, and wherein the instructions for administering the composition provide instructions to a user of the kit to administer the FAK inhibitor at the concentration in the large mammal.

2. The kit of claim 1, wherein the composition is adapted to reduce fibroblast proliferation in tissues of large mammals and promote tissue regeneration, and/or the FAK inhibitor modulates mechanical signaling and promotes tissue regeneration.

3. (canceled)

4. The kit of claim 1, wherein the porous scaffold hydrogel is a thin film.

5. The kit of claim 1, wherein the FAK inhibitor is selected from one or more of the group consisting of VS-6062, PF-562271, PR-573228, NVP-TAE226, PF-03814735, PF-562271 HCl, GSK2256098, PF-431396, VS-4718, VS-6063, PF-04554878, and Nonaisoprenol.

6. The kit of claim 1, wherein the kit further comprises a wound dressing configured to protect the wounded tissue.

7. A method of promoting tissue healing while reducing fibrosis in a large mammal, comprising:
disposing a composition comprising an effective amount of a focal adhesion kinase (FAK) inhibitor disposed in pores of a pullulan-collagen hydrogel scaffold in proximity to a tissue of the large mammal having, an adult weight of greater than about 7 kilograms, wherein the tissue comprises a wound;
dispensing the FAK inhibitor from the composition into the proximity of the wounded tissue; and
reducing a level of focal adhesion kinase for a selected period of time, thereby reducing fibrosis while healing the wounded tissue.

8. (canceled)

9. The method of claim 7, wherein the composition comprising the FAK inhibitor is administered locally.

10. (canceled)

11. The method of claim 7, wherein the pullulan-collagen hydrogel is a thin film.

12-13. (canceled)

14. The method claim 7, wherein the FAK inhibitor is selected from one or more of the group consisting of VS-6062, PF-562271, PR-573228, NVP-TAE226, PF-03814735, PF-562271 HCl, GSK2256098, PF-431396, VS-471, VS-6063, PF-0455487878, Nonaisoprenol.

15. The method of claim 7, wherein the wound is an incision, a penetrating wound, or a burn.

16. The method of claim 7, wherein the FAK composition is formulated for controlled release of the FAK inhibitor.

17. The method of claim 7, wherein the selected period of time for treatment with the composition comprising the FAK inhibitor is from about 7 days to about 100 days.

18. The method of claim 7, wherein the composition comprising the FAK inhibitor is freshly applied to the proximity of the tissue every 36 to 48 hours.

19. The method of claim 7, wherein the effective amount of the focal adhesion kinase inhibitor is from 30-100 micrograms/g tissue by weight.

20. The kit of claim 1, wherein the instructions instruct the user to freshly apply the composition to the wounded tissue every 36 to 48 hours, or the instructions instruct the user to freshly apply the composition to the wounded tissue from about 7 days to about 100 days after an initial administration.

21. The method of claim 7, wherein the composition is adapted to reduce fibroblast proliferation in tissues of large mammals and promote tissue regeneration, and/or the FAK inhibitor modulates mechanical signaling and promotes tissue regeneration.
